(19) United States
(12) Patent Application Publication
(10) Pub. No.: US 2025/0029701 A1
(43) Pub. Date: Jan. 23, 2025

(54) METHODS OF REGULATING ACCESS TO QSYMIA TO MITIGATE POTENTIAL DRUG-ASSOCIATED RISKS

(71) Applicant: VIVUS LLC, Campbell, CA (US)

(72) Inventors: Michael MILLER, Menlo Park, CA (US); Barbara TROUPIN, Red Bluff, CA (US)

(21) Appl. No.: 18/906,278

(22) Filed: Oct. 4, 2024

Related U.S. Application Data
(63) Continuation of application No. 18/356,421, filed on Jul. 21, 2023, now abandoned.
Application No. 18/356,421 is a continuation of application No. 17/672,314, filed on Feb. 15, 2022, now abandoned.
Application No. 17/672,314 is a continuation of application No. 16/732,508, filed on Jan. 2, 2020, now abandoned.
Application No. 16/732,508 is a continuation of application No. 16/008,808, filed on Jun. 14, 2018, now abandoned.
Application No. 16/008,808 is a continuation of application No. 15/795,073, filed on Oct. 26, 2017, now abandoned.
Application No. 15/795,073 is a continuation of application No. 15/464,061, filed on Mar. 20, 2017, now abandoned.
Application No. 15/464,061 is a continuation of application No. 14/213,498, filed on Mar. 14, 2014, now abandoned.
(60) Provisional application No. 61/785,313, filed on Mar. 14, 2013.

(51) Int. Cl. G16H 20/13; G09B 19/00; G09B 23/28; G16H 20/10; G16H 40/00

(52) U.S. Cl. CPC G16H 20/13; G09B 19/00; G09B 23/28; G16H 20/10; G16H 40/00

(57) ABSTRACT

The present invention provides methods for dispensing or distributing a drug, particularly a potentially teratogenic drug, to a patient while minimizing the occurrence of an adverse side effect. In particular, the present invention provides methods for dispensing or distributing a drug containing topiramate to a patient in need of weight loss to minimize the occurrence of potential birth defects associated with the drug. Methods for providing training to prescribers on the potential risks associated with particular drugs are also disclosed.

### CROSS-REFERENCE TO RELATED APPLICATIONS

This application is a continuation of U.S. application Ser. No. 18/356,421, filed Jul. 21, 2023, which is a continuation of U.S. application Ser. No. 17/672,314, filed Feb. 15, 2022, which is a continuation of U.S. application Ser. No. 16/732,508, filed Jan. 2, 2020, which is a continuation of U.S. application Ser. No. 16/008,808, filed Jun. 14, 2018, which is a continuation application of U.S. application Ser. No. 15/795,073, filed Oct. 26, 2017, which is a continuation application of U.S. application Ser. No. 15/464,061, filed Mar. 20, 2017, which is a continuation application of U.S. application Ser. No. 14/213,498, filed Mar. 14, 2014, which claims priority to U.S. Provisional Application No. 61/785,313 filed Mar. 14, 2013, each of which is hereby incorporated by reference in its entirety.

### FIELD OF THE INVENTION

The present invention relates to methods for distributing a drug to a patient to minimize serious adverse effects associated with the drug. In particular, the present invention provides a method for reducing or alleviating the potential risks of serious adverse effects to a fetus associated with dispensing a drug with teratogenic properties to a patient.

### BACKGROUND OF THE INVENTION

Many beneficial drugs on the market today have the potential to produce serious adverse effects in some patient populations. Although these drugs can be administered safely to most patients, they are contraindicated in specific patients due to an individual patient's medical condition, medical history, and/or concurrent treatment with other drugs. Procedures are necessary to distribute such drugs to the appropriate patient population while minimizing or avoiding distribution to patients for whom the drug is contraindicated.

To ensure a drug's benefits outweigh its risks, the U.S. Food and Drug Administration (FDA) can require that particular risk evaluation and mitigation strategies (REMS) be established by the drug manufacturer. Such strategies are indicated where patient education regarding the potential risks of using the drug and patient adherence to directions for use are critical for the drug's effectiveness. However, several factors must be considered when determining the appropriate number and type of mitigation procedures to employ to safely dispense drugs associated with serious potential adverse effects. For instance, appropriate mitigation procedures will balance the severity of the potential adverse effect and its probability of occurrence with the demand created by the procedures on pharmacies, prescribers, and patients and the ease with which the participating parties can comply with the procedures. Such mitigation procedures must be tailored to each specific drug taking into account the factors described above as well as the feasibility of implementing and monitoring the efficacy of such procedures in controlling the distribution of the drug.

One class of drug that warrants the establishment of effective procedures to control distribution is teratogenic drugs, which have the potential to cause birth defects in fetuses in women who are pregnant or become pregnant while taking the drug. However, even within this single category of drugs, there is considerable variability as to the severity and frequency of occurrence of particular potential birth defects that necessitates procedures employing different levels of control. For example, thalidomide is a proven teratogen that causes serious birth defects and therefore, controls restricting both access and distribution of the drug are required. On the other hand, for drugs that are suspected teratogens (increased risk but not proven to cause birth defects), procedures that provide patient and prescriber education and monitoring of distribution of the drug may be sufficient to minimize the occurrence of potential birth defects.

Accordingly, there is a need in the art to develop new mitigation strategies and procedures to safely dispense to a patient drugs associated with serious potential adverse effects to ensure that the benefits of the drug outweigh its risks. In particular, there is a need for additional checks and controls to ensure that a drug with potential teratogenic properties is not administered to patients for whom the drug is contraindicated, such as pregnant women or women who can become pregnant during drug treatment.

### SUMMARY OF THE INVENTION

The present invention provides a method for dispensing a drug (e.g. teratogenic drug) safely to patients while minimizing the occurrence of a potential adverse side effect in specific patient populations. In one particular embodiment, the present invention provides a method for dispensing a drug containing topiramate, optionally in combination with phentermine, to a patient in need of weight loss (e.g. obese or overweight patient with a co-morbidity) while ensuring that the drug is not administered to patients for whom the drug is contraindicated.

In one embodiment, the method comprises identifying a population of eligible pharmacies and certifying each pharmacy to obtain a population of certified pharmacies. Preferably each certified pharmacy will agree to and comply with the following requirements: (i) purchase the drug only from the supplier or authorized distributor; (ii) dispense the drug only from selected certified locations that are approved by the supplier; (iii) refrain from reselling or transferring the drug to any other pharmacy or distributor; (iv) train appropriate staff on the requirement to distribute specified educational material with each prescription of the drug; (v) implement and maintain a pharmacy management system to ensure that specified educational material is provided with each prescription each time the drug is dispensed; (vi) be subject to periodic audits; and (vii) collect prescriber data for each prescription and provide that data to the supplier. Pharmacies that are eligible to be certified pharmacies will have an established data management system to collect prescriber data for prescribers of the drug and to direct distribution of information related to the risks associated with taking the drug to the patient each time a prescription for the drug is filled. The population of eligible pharmacies can be registered in a computer readable storage medium following certification and provided with information related to the potential risks associated with taking the drug. Distribution of the drug is authorized to certified pharmacies, which in turn dispense the drug pursuant to a prescription to the patient with information related to the potential risks associated with taking the drug. In some embodiments, the certified pharmacies dispense the drug to the patient only by mail order. In other embodiments, the certified pharmacies dispense the drug to the patient through select, certified retail dispensing locations.

The information related to the potential risks associated with taking the drug can include a medication guide and/or a patient brochure or other approved patient information. In some embodiments, the medication guide and/or the patient brochure is provided to the patient with each new prescription and each refill of the drug. The medication guide and/or the patient brochure include information relating to the potential risk of birth defects associated with the drug, the need for pregnancy prevention before and during drug treatment, and directions to immediately discontinue use of the drug if the patient were to become pregnant. In certain embodiments, the birth defect is a congenital malformation, including orofacial clefts (e.g., cleft lip and/or cleft palate).

In another embodiment, the methods of the present invention comprise contacting prescribers of the drug and providing voluntary training to the prescribers on the proper use of the drug and the potential risks associated with the drug. Prescribers who complete the training, can be registered in a computer readable storage medium as trained prescribers. In some embodiments, a list of the registered prescribers that have completed training is compared to a list of prescribers of the drug to identify unregistered prescribers who have not completed training. Such unregistered (i.e. not-yet-trained) prescribers may be contacted and provided with training or access to training materials. At least 95% or greater and preferably all prescribers of the drug are contacted and offered training on the potential risks associated with the drug and guidance on safe use.

### BRIEF DESCRIPTION OF THE DRAWINGS

FIG. 1 is a flowchart describing a method for providing the drug to patients and building a database on prescribing health care providers (“HCPs”) by having certified pharmacies capture and provide the data to a central location.

FIG. 2 is a flowchart describing a method of pharmacy certification.

FIG. 3 is a flowchart describing a method for training health care providers about the potential risks of the drug by comparing prescriber data to trained prescriber (HCP) data.

FIG. 4 is a flowchart describing a method for determining if a patient is an appropriate candidate for the drug and whether the patient should be provided additional educational material or counseling.

### DETAILED DESCRIPTION OF THE INVENTION

### Definitions and Nomenclature

It must be noted that, as used in this specification and the appended claims, the singular forms “a,” “an” and “the” include plural referents unless the context clearly dictates otherwise. Thus, for example, “an active agent” refers not only to a single active agent but also to a combination of two or more different active agents, “a dosage form” refers to a combination of dosage forms as well as to a single dosage form, and the like.

Unless defined otherwise, all technical and scientific terms used herein have the meaning commonly understood by one of ordinary skill in the art to which the invention pertains. Specific terminology of particular importance to the description of the present invention is defined below.

When referring to an active agent, applicants intend the term “active agent” to encompass not only the specified molecular entity but also its pharmaceutically acceptable, pharmacologically active analogs, including, but not limited to, salts, esters, amides, prodrugs, conjugates, active metabolites, and other such derivatives, analogs, and related compounds as will be discussed infra. Therefore, reference to “phentermine” encompasses not only phentermine per se but also salts and other derivatives of phentermine, e.g., phentermine hydrochloride. It is to be understood that when amounts or doses of phentermine are specified, that those amounts or doses refer to the amount or dose of phentermine per se and not to a phentermine salt or the like. For example, when it is indicated that a dose or amount of phentermine is 3.75 mg, that would correspond to 4.92 phentermine hydrochloride and not 3.75 phentermine hydrochloride.

The terms “treating” and “treatment” as used herein refer to reduction in severity and/or frequency of symptoms, elimination of symptoms and/or underlying cause, and improvement or remediation of damage. In certain aspects, the term “treating” and “treatment” as used herein refer to the prevention of the occurrence of symptoms. In other aspects, the term “treating” and “treatment” as used herein refer to the prevention of the underlying cause of symptoms associated with obesity, excess weight, and/or a related condition. The phrase “administering to a subject” refers to the process of introducing a composition or dosage form of the invention into the subject (e.g., a human or other mammalian subject) via an art-recognized means of introduction.

By the terms “effective amount” and “therapeutically effective amount” of an agent, compound, drug, composition or combination of the invention which is nontoxic and effective for producing some desired therapeutic effect upon administration to a subject or patient (e.g., a human subject or patient).

The term “dosage form” denotes any form of a pharmaceutical composition that contains an amount of active agent sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics, such as hydrophilicity.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release, modified release, and delayed release formulations.

The term “sustained release” (synonymous with “extended release”) is used in its conventional sense to refer to a drug formulation that provides for gradual release of a drug over an extended period of time, and that preferably, although not necessarily, results in substantially constant blood levels of a drug over an extended time period and a lower peak blood level than immediate release. The term “delayed release” is also used in its conventional sense, to refer to a drug formulation which, following administration to a patient, provides a measurable time delay before drug is released from the formulation into the patient's body.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “subject” or “individual” or “patient” refers to any subject for whom or which therapy is desired, and generally refers to the recipient of the therapy to be practiced according to the invention. The subject can be any vertebrate, but will typically be a mammal. If a mammal, the subject will in many embodiments be a human, but may also be a domestic livestock, laboratory subject or pet animal.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### Methods of the Invention

The present invention is directed generally to methods for distributing or dispensing a drug to a patient while minimizing the occurrence of an adverse side effect known to be or suspected of being associated with administration of the drug. As used herein, “drug” refers to any substance which is intended for use in the diagnostics, cure, mitigation, treatment, or prevention of disease, or to affect the structure or function of the human body. In certain embodiments, the drug contains topiramate.

The methods described herein may be advantageously employed to avoid distribution of one or more drugs known or suspected of causing an adverse side effect to a patient for whom the drugs may be contraindicated. As used herein, an “adverse side effect” refers to any abnormality, defect, mutation, lesion, degeneration, or injury, which may be caused by taking the drug. The adverse side effect may occur in the patient receiving the drug or in a fetus carried by the patient receiving the drug. Thus, the methods of the present invention may be used to minimize the occurrence of one or more adverse side effects associated with a drug. The term “minimize,” as used in this context generally means that there is an avoidance rate in observing adverse side effects of greater than about 60%, about 65%, about 70%, about 75%, about 80%, about 85%, about 90%, or about 95%. In certain embodiments, a drug may be dispensed to a patient with substantially no observation of adverse side effects (i.e. nearly or up to a 100% avoidance rate).

As used herein, the term “contraindicated” refers to any condition in a patient which renders a particular line of treatment, including the administration of one or more drugs, undesirable or improper. This condition may be preexisting, or may develop while the patient is taking the drugs, including conditions which may result directly or indirectly from treatment with the drugs. Drugs may also be considered “contraindicated,” as the term is used herein, if use of a drug by patients who are also taking another drug is known or suspected of producing an adverse side effect in those patients. In one embodiment, the drug comprises topiramate optionally in combination with phentermine. Topiramate is contraindicated in certain patient populations, including, but not limited to, patients who are pregnant, suffering from or diagnosed with glaucoma, suffering from or diagnosed with hyperthyroidism, taking monoamine oxidase inhibitors, or have a known hypersensitivity or idiosyncrasy to sympathomimetic amines.

The methods of the present invention are particularly useful for the distribution of potentially teratogenic drugs, such as topiramate, the administration of which is associated with the observation of one or more adverse side effects. “Teratogenic drugs” refer to drugs that are capable of interfering with the development of a fetus that may lead to potential birth defects or developmental malformations. In some embodiments, the present invention provides a method for dispensing or distributing a drug containing topiramate to a patient while minimizing the occurrence of an adverse side effect. Adverse side effects associated with topiramate include, but are not limited to, birth defects, increase in suicidal thoughts and behavior, acute myopia with secondary angle closure glaucoma, increased heart rate, mood disorders (e.g., depression and anxiety), sleep disorders (e.g., insomnia), cognitive impairment (e.g., impairment of concentration and/or attention, difficulty with memory, and speech or language problems), and metabolic acidosis. In one particular embodiment, the methods of the present invention minimize the occurrence of congenital malformations in infants, including orofacial clefts, such as cleft lip and cleft palate.

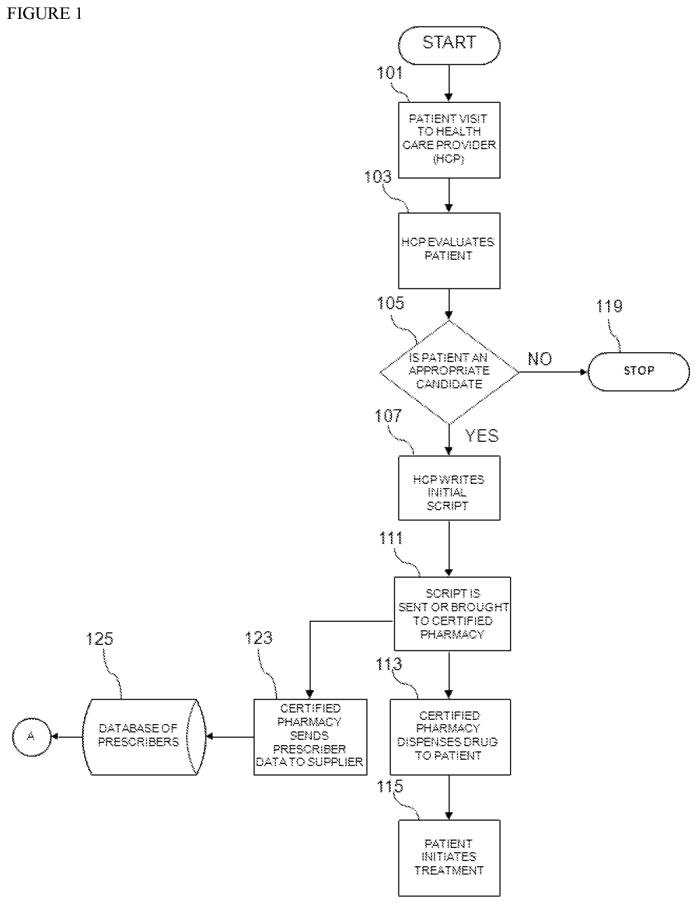

FIG. 1 represents a high level work flow for a process of providing the drug to patients according to one embodiment of the invention. The patient visits a health care provider (HCP) 101 and is evaluated by the HCP 103. The HCP determines that the patient is either an appropriate candidate for the drug or not an appropriate candidate for the drug 105. If the patient is not an appropriate candidate the HCP does not prescribe the drug 119. If the HCP determines that the patient is an appropriate candidate, the HCP may write one or more initial prescriptions for the drug 107. The prescription(s) is sent, by the HCP or the patient, or brought by the patient to the certified pharmacy, 111. The certified pharmacy collects the prescriber data from the prescription and may be required to call the prescriber to obtain complete information that is then provided to the manufacturer of the drug (supplier) or a designated third party 123. If all necessary information has been collected and conforms to the policy, the certified pharmacy fills the prescription and dispenses the drug to the patient along with the medication guide and patient brochure 113. The patient may receive the drug through the mail or from select, certified retail locations. When the patient receives the drug they may initiate treatment immediately 115. The prescriber information that was sent to the manufacturer (supplier) or designated third party in step 123 is then added to a database of prescribers 125.

In certain embodiments, the drug distribution methods of the present invention comprise identifying a population of pharmacies that are eligible to dispense the drug. Pharmacies that are eligible to dispense the drug include pharmacies that have an established management system (e.g. software-based or otherwise) capable of maintaining a list of prescribers of the drug and directing the distribution of information (e.g., information related to the risks associated with taking the drug) to the patient each time a prescription for the drug is filled. In some embodiments, eligible pharmacies agree, for example by contract or attestation, to (a) refrain from reselling or transferring the drug to another pharmacy or distributor, (b) train employees on the risks associated with taking the drug and the proper measures for dispensing the drug, and/or (c) submit to periodic audits of procedures for dispensing the drug. Eligible pharmacies may, in some embodiments, also agree to one or more of the following: (a) to purchase product only from the supplier (e.g., drug manufacturer) or a third party designated by the supplier; (b) to ensure that product will be dispensed only by selected, certified locations; (c) to provide prescriber and/or patient data to the supplier or a third party designated by the supplier; and/or (d) to dispense the drug in no more than 30-day quantities. In certain embodiments, eligible pharmacies are mail order pharmacies.

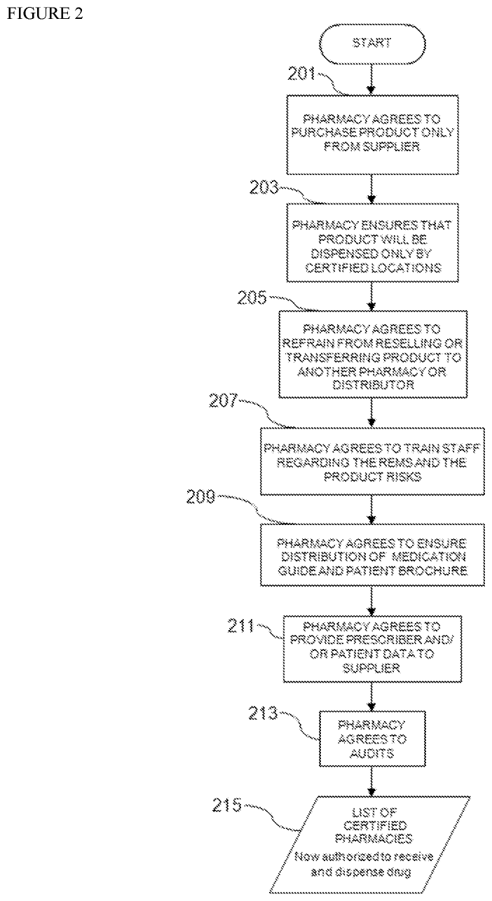

FIG. 2 is a workflow for one method of certifying a pharmacy according to one embodiment of the present invention. The pharmacy to be certified agrees to purchase product only from the supplier (e.g., drug manufacturer) or a third party designated by the supplier 201. The pharmacy agrees to ensure that product will be dispensed only by certified locations 203. The pharmacy agrees that it will not resell or transfer the drug to any other pharmacy or distributor 205. The pharmacy agrees to train staff regarding the requirements for product (e.g. drug) distribution and the product risks 207. In some aspects the pharmacy identifies one or more individuals that will be responsible for overseeing the filling of prescriptions for the drug and that individual(s) certifies in writing that he or she will adhere to the specified REMS certification requirements. The pharmacy agrees that it will ensure distribution of a medication guide and patient brochure specific for the product with each prescription that is filled 209. The pharmacy agrees to provide prescriber and/or patient data to the supplier or a third party designated by the supplier 211. In some aspects, the data to be gathered and provided by the certified pharmacy includes at least a subset of the data shown in Tables 1, 2, and 3 infra. The pharmacy agrees to periodic audits by or on behalf of the supplier to determine compliance with the certification requirements 213. Audits may be, for example, yearly, every other year or twice yearly of each certified pharmacy or a subset of certified pharmacies. The frequency of audits may change over time, for example, yearly in the first or first several years and every other year in subsequent years. Data may be provided weekly, monthly, on a varied schedule or on demand. If pharmacies are found to be non-compliant they may be decertified or the supplier (e.g., drug manufacturer) may take corrective action to bring the pharmacy back into compliance. Pharmacies that agree to each of the requirements may be certified by the supplier to obtain and dispense the drug and are added to a list of certified pharmacies 215. Although the requirements for certification are shown in an ordered flow chart it will be understood that there is no particular order required for the steps. In other embodiments, pharmacies may be obligated to agree to additional requirements, such as dispensing the drug in no more than 30-day quantities.

Pharmacies that meet the eligibility criteria as described herein are preferably registered in a computer readable storage medium, such as a database or internet website, to obtain a record of certified pharmacies that are authorized to obtain and dispense the drug. Other suitable computer readable storage media which may be employed for registration of the pharmacies (as well as the prescribers, as discussed below) will be apparent to one of ordinary skill in the art, once armed with the teachings of the present application. In some embodiments, the list of registered, certified pharmacies is maintained in a database or on an internet website that is accessible by the public. In some instances, it may be desirable to limit the number of eligible pharmacies that can be certified and registered. For example, in certain embodiments, ten or fewer eligible pharmacies are certified and registered. In other embodiments, five or fewer eligible pharmacies are certified and registered. In still other embodiments, two to five eligible pharmacies are certified and registered. Certified pharmacies may, in some embodiments, dispense drug from only a single location. In other aspects, certified pharmacies may dispense drug from multiple locations. For instance, in some aspects, retail locations that are affiliated with the certified pharmacies (e.g. retail dispensing locations) are certified to distribute the drug directly to patients through in-store pick-up as described in more detail infra. The drug manufacturer may provide a web-based application to assist patients in locating a certified pharmacy or certified retail dispensing location.

The registration of the eligible pharmacy may be achieved by providing the pharmacy, for example, by mail, facsimile transmission, or on-line transmission, with a registration card or form. The pharmacy may then have the registration card or form completed by providing the information requested therein, which thereafter may be returned to the manufacturer or distributor of the drug, or other authorized recipient of the registration card or form, for example, by mail, facsimile transmission or on-line transmission. Information which may be requested of the pharmacy in the registration card or form may include, for example, the pharmacy's name, address, and affiliation, if any, with any health care institution such as, for example, a hospital, health care organization, and the like. The pharmacy's information in the registration card or form is then preferably entered into the computer readable storage medium (e.g., database or internet website). In certain preferred embodiments, the pharmacy is registered using a unique identifier, such as a Drug Enforcement Administration (DEA) and/or a National Council for Prescription Drug Programs (NCPDP) identification number.

Retail pharmacy locations (or retail dispensing locations) of a certified pharmacy that have an affiliated retail pharmacy corporate office that serves as the authorized representative for all retail pharmacies in a single corporation may be certified to dispense the drug directly to patients. In certain embodiments, the authorized representative configures and implements a pharmacy management system, verifies connectivity of each retail pharmacy location (retail dispensing location) with a database support center (or “switch provider”), implements policies and procedures to support compliance with the certification requirements and provides training materials to the retail dispensing locations.

To become certified, each retail dispensing location (1) completes pharmacy training and knowledge assessment on the risks associated with taking the drug and the proper measures for dispensing the drug, (2) agrees to comply with specified requirements of the drug manufacturer, (3) provides complete information about the location, and (4) provides evidence of completion of these steps to the affiliated retail pharmacy corporate office (or other contracted entity). The pharmacy training may include a description of the dispensing requirements for the drug (e.g., distribution of educational materials to the patient each time the drug is dispensed), information about the drug, the potential risk of teratogenicity associated with the drug, the need to counsel female patients about pregnancy prevention, the recommendation for pregnancy testing prior to initiation and during treatment with the drug, and the need to discontinue use of the drug if pregnancy occurs. The pharmacy training may be in the form of a series of slides with questions to assess knowledge of the information presented. In some embodiments, the training is presented in electronic format and may be integrated into the training program for the retail pharmacy location. A link to a restricted access web site may be provided to pharmacies to provide access to the training.

In some embodiments, once a lead pharmacist at the retail dispensing location completes training and knowledge assessment successfully, a completion code will be generated and provided to the pharmacist. The pharmacist may use the code to complete the certification process for the retail dispensing location by providing the required information about the location and completing the necessary attestation to comply with the requirements of the drug manufacturer. In such embodiments, the pharmacist may be required to attest to one or more of the following: (a) all dispensing staff within the dispensing location are trained on the risks associated with taking the drug and the proper measures for dispensing the drug, and appropriate documentation of such training is maintained, (b) he/she understands the pharmacy management system configuration and that required connectivity has been successfully verified by the drug manufacturer, (c) all prescription claims for the drug, regardless of the method of payment, must be processed through the pharmacy management system and claims routing switch, and (d) the location must comply with periodic surveys or audits. Upon completion of the certification process, the individual retail dispensing locations are then added to the database of registered, certified pharmacies and are subsequently authorized to obtain the drug from the manufacturer or authorized distributor and to dispense the drug to patients having a prescription. Preferably, the certification status of the pharmacy or its retail dispensing location is verified by the drug manufacturer or authorized distributor prior to making each shipment of drug.

Certified retail dispensing locations may be audited or surveyed periodically to ensure that the conditions for restricted distribution are being met. For example, the drug manufacturer may survey about 25% of the certified dispensing locations each year and perform audits of a sample of pharmacies that fail to respond to the survey, perform below a selected level on the survey responses or provide inadequate or untimely internal audit information. The drug manufacturer may also monitor distribution data and healthcare provider level prescription data to identify events of non-certified pharmacies acquiring or dispensing the drug.

Where a certified pharmacy has a number of chain retail dispensing locations a survey of about 3%, or about 5%, or about 5-10% of all chain retail dispensing locations annually may be used to assess compliance. Surveys for chain locations that are affiliated with a corporate entity may be directed through the corporate entity. Independent retail dispensing locations may similarly be surveyed randomly at levels of about 3%, or about 5%, or about 5-10% of all independent retail dispensing locations annually. Locations that do not respond to the survey request within about 30 days will be contacted again and encouraged to complete the survey. Locations that answer the survey questions with less than about 70% accuracy will be identified. In the case of chain locations of a corporate pharmacy, low performing (e.g. less than about 70% accuracy) locations may be reported to the corporate entity. For independent locations, low performers will be contacted and provided retraining. Continued low performance may result in decertification.

Patient surveys may also be used to measure compliance with the requirements. Patients may be recruited through prescriber lists and given an incentive to participate or to complete the survey. Patients may be selected independent of the type of pharmacy used for dispensing or may be selected based on pharmacy type. For example, about 250, or about 200-500 or more patients may be sent surveys independent of pharmacy type or a number of patients, e.g. about 200 or more, can be selected from each chain and independent retail pharmacies. Patients may be asked if they received selected materials or may be asked to describe all of the materials received from the pharmacy.

Assessment of patient knowledge and understanding may also be surveyed periodically in a representative sample of patients taking the drug that are females of reproductive potential. The survey will seek to determine information regarding receipt of the medication guide and patient brochure as well as the effectiveness of the materials in communicating the risks of teratogenicity associated with the drug. The extent of counseling about pregnancy prevention and contraceptive use may be assessed as well as the use of contraceptives by females of reproductive potential. Preferably the sample size will be at least about 250 patients annually. The same patients may be surveyed in subsequent years or different patients may be selected.

Assessment of the effectiveness of the methods may be conducted by the drug manufacturer every 6 or 12 months and may include the following (i) information regarding certified pharmacy failure to adhere to dispensing requirements, (ii) information regarding dispensing that occurs outside of the certified pharmacies, (iii) evaluation of any modifications to the program that may result in improved compliance and achievement of increased safe use, and (iv) a listing of pharmacies that have been decertified and reasons for decertification. Information regarding compliance by distributors may also be collected, including quarterly distributor compliance reports, report of any distribution of the drug to non-certified pharmacies and the names of distributors that have been excluded from being able to distribute and the reasons for exclusion.

In preferred embodiments, each retail pharmacy corporate office will designate an authorized representative to assume responsibility for coordinating compliance within that organization and to complete the corporate attestation for the organization. A lead pharmacist may be designated at each dispensing location and the lead pharmacist may be tasked with overseeing pharmacy staff training and compliance with the drug distribution requirements at that location. Standard methods of communication between pharmacies and payers will be used so as not to disrupt the standard prescription fulfillment workflow in the retail pharmacy environment, for example, pharmacy management systems may communicate through switch providers as is the normal course of pharmacy dispensing. A switch provider provides information to pharmacies at point-of-dispensing via their pharmacy management system terminals. The system will notify the pharmacy if required data is missing and will prompt the pharmacy to submit the data prior to dispensing. In certain embodiments, all prescription claims for the drug, regardless of payment method, are routed through the switch provider. A claim rejection notification will be provided to pharmacies that are not certified but attempt to dispense the drug. In some embodiments, the system may prompt the pharmacy to provide specific educational materials (medication guides and/or patient brochures) to patients with every prescription dispensed.

The drug manufacturer may enter into a written agreement or contract with a distributor, distribution center, warehouse, or wholesaler (collectively referred to as authorized distributors) that are capable of meeting the restricted distribution requirements. Authorized distributors may designate an individual to be the authorized representative to be responsible for internally coordinating and overseeing the distribution of the drug and compliance with the distribution requirements for the drug. The role of the authorized representative may include (a) ensuring systems, protocols or other processes are configured and in place to ensure distribution conditions are met; (b) assure all persons involved in the distribution systems are trained to verify a pharmacy's certification status prior to distributing the drug to a certified pharmacy or certified retail dispensing location; (c) provide quarterly compliance reports to the manufacturer; and (d) make data available to the drug manufacturer in a timely fashion in the event of random verification audits, which may occur quarterly or may be random.

The drug manufacturer may facilitate pharmacist awareness of the restricted distribution program for the drug by providing written communications to retail pharmacy dispensing locations, for example, such as those affiliated with mail order pharmacies that are already distributing the drug or to pharmacies that have identified capabilities consistent with having the capacity to become a certified pharmacy. The communication may provide the goals and requirements of the restricted distribution program and the risks associated with the drug. The communication may be in the form of a letter that may be sent by mail, fax or electronic mail. The letter may include one or more links to websites that provide additional information or training in connection with the prescription of the drug. The manufacturer may establish a support center to provide support to the authorized distributors and certified pharmacies.

In order to maintain registration (certification), certified pharmacies may be obligated to comply with one or more requirements including, conducting internal audits of management systems and procedures for dispensing the drug and providing periodic (e.g. quarterly) compliance reports to the drug manufacturer or authorized distributor. Certified pharmacies may, on a periodic basis, be required to certify that (a) a pharmacy management system is in place to direct that information related to the risks associated with the drug is provided to the patient each time the drug is dispensed, (b) the pharmacy has not resold or transferred the drug to another unregistered pharmacy, distributor or other outlet, (c) the pharmacists dispensing the drug have been trained regarding the risks associated with the drug and the need to provide such information to the patient, and/or (d) the pharmacy is maintaining a list of prescribers of the drug. Certified pharmacies preferably agree that they will obtain drug only from the supplier or the supplier's authorized distributor. Certified pharmacies that do not fulfill these requirements are subject to loss of certified status and are no longer considered to be “certified pharmacies” authorized to dispense the drug to the patient. In such circumstances, the drug manufacturer may provide access to re-training or implement a corrective action plan and perform a follow-up audit to verify compliance. If a pharmacy is not able to comply after these measures, the pharmacy may be barred from further distribution of the drug.

According to certain embodiments, the methods of the invention comprise providing certified pharmacies with educational materials to ensure proper dispensing of the drug according to the methods described herein, including, for example, information related to the risks associated with taking the drug for which the pharmacy is registered/certified to dispense as well as suitable methods for distributing the drug to the patient. A wide variety of educational materials may be employed to ensure proper dispensing according to the methods described herein, including, for example, various forms of literature materials. The term “literature,” as used herein, refers to a variety of materials including, for example, product information, letters to pharmacists (e.g. Dear Pharmacist Letter), letters to health care providers (e.g. Dear Healthcare Provider Letter), letters to professional societies or organizations affiliated with healthcare (e.g. Dear Professional Society Letter), educational brochures, patient brochures, medication guides, continuing education monographs, videotapes and the like, and various combinations of two or more of such materials. Depending on the nature and the type of the involved materials, the literature may be paper-based or may be in electronic format. The Dear Healthcare Provider letter may be sent out on multiple occasions, for example, within sixty days of drug approval and again at 12 and 24 months after drug approval. It may initially be sent to healthcare providers that have written a prescription for a medical treatment for indications related to those that may be treated with the drug (e.g. obesity treatments) within a prior period of time, for example, the prior 12 months. A Dear Professional Society Letter may be sent to medical societies and may request that the Dear Healthcare Provider Letter be sent to the members of the professional society. In some embodiments, the Dear Pharmacist Letter and pharmacist training materials may be provided only to pharmacies that have demonstrated an ability to comply with the requirements of being a certified pharmacy.

In certain embodiments, information related to the risks associated with taking the drug comprises a medication guide and/or a patient brochure. The medication guide and/or patient brochure may be separate documents or may be combined into a single document and can include information on adverse side effects associated with the drug and instructions to the patient on proper use of the drug. In embodiments in which the drug is a potential teratogenic drug, such as topiramate, the medication guide and/or the patient brochure can include information such as a description of the potential risk of birth defects associated with taking the drug, the need to prevent pregnancy while taking the drug (e.g., through use of one or more contraceptives), the importance of pregnancy testing before and during the drug treatment period, and directions to immediately discontinue taking the drug upon becoming pregnant. In one particular embodiment, the medication guide and/or a patient brochure describes the potential risk of congenital malformations in infants, such as orofacial clefts (cleft lip and/or cleft palate), associated with exposure to topiramate. In preferred embodiments, a medication guide and/or a patient brochure is provided to patients by certified pharmacies with each new prescription of the drug and each refill. The medication guide and/or patient brochure can, in some embodiments, be available on the internet, by calling a toll free number, and/or by delivery by field-based personnel, such as medical liaisons and representatives of the drug manufacturer or authorized distributor.

In further embodiments of the invention, once eligible pharmacies are certified in the computer readable storage medium and provided with information related to the potential risks associated with the drug for which they are certified to dispense, the certified pharmacies may be authorized to receive shipment of the drug. After receiving shipment of the drug, the certified pharmacies dispense the drug pursuant to a prescription to the patient with the information related to the potential risks associated with taking the drug. Preferably, such information (e.g., a medication guide and patient brochure) should be provided to the patient with each new prescription of the drug and each refill. In one embodiment, the certified pharmacies have an automated management system that instructs pharmacists and staff dispensing the drug to include information related to the potential risks associated with taking the drug (e.g., a medication guide and patient brochure) with each prescription and each refill of the drug.

In certain embodiments, the certified pharmacies receive the prescription for the drug from the patient or the prescriber through the mail or facsimile. In related embodiments, the certified pharmacies dispense the drug to the patient only by mail order. In other embodiments, certified pharmacies receive the prescription for the drug from the patient at certified retail dispensing locations and dispense the drug to the patient in person. In some aspects distribution over the internet is restricted or not allowed and pharmacies are not permitted to resell or sample the drug. In other aspects the use of stock bottles by pharmacies is prohibited. In some embodiments, the amount of drug which is prescribed to the patient is for a limited amount with a limited number of refills. For instance, in certain embodiments, the drug is dispensed in a supply not to exceed thirty days. In other embodiments, the number of refills is limited to five or less.

Although many of the method steps disclosed herein serve to minimize the occurrence of adverse side effects, one of skill in the art will appreciate that the steps may also result in making patient access to the drug more difficult. If similar products are available that are not burdened by the restricted access methods disclosed herein it may be beneficial to take steps to limit such access in order to achieve the desired reduction in adverse side effects. For example, in the case of a combination drug product for which the individual active ingredients may be available through off-label prescription through retail pharmacies, if access to the approved product is too difficult patients may seek to obtain prescriptions to the individual components if they are more easily accessible. If the individual off-label prescriptions are filled without any of the methods to regulate access or requirements to provide educational material about the risks of the drug or drugs adverse side effects may result. To mitigate this effect, steps may be taken to reduce the burden on patients, for example, increasing the number of certified pharmacies and providing a web based or mobile application for locating certified pharmacies.

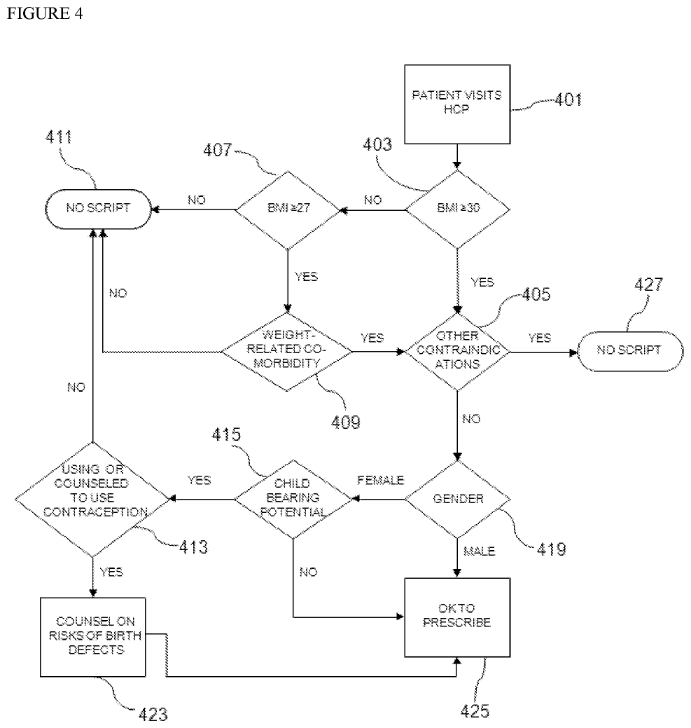

In accordance with embodiments of the present invention, the methods described herein may be particularly useful for dispensing a drug containing topiramate (e.g. an extended release form of topiramate) to a patient in need of weight loss. A patient in need of weight loss can include a patient who has a body mass index (BMI) of 25 kg/m² or greater, 27 kg/m² or greater, 30 kg/m² or greater, 35 kg/m² or greater or 40 kg/m² or greater. A patient in need of weight loss can also include a patient who has one or more weight-related morbidity factors, which can include, but are not limited to, hypertension, type 2 diabetes mellitus, or dyslipidemia. In some embodiments, a patient in need of weight loss is a patient who has a BMI of 27 kg/m² or greater in the presence of at least one weight-related morbidity factor. In certain embodiments, a patient in need of weight loss is a female of reproductive potential or child bearing potential. As used herein, “a female of reproductive potential or child bearing potential” refers to a female who has never had a hysterectomy, surgical sterilization, both ovaries removed (e.g., bilateral oophorectomy), medically documented spontaneous ovarian failure, or has not gone through menopause. Preferably, the patient in need of weight loss is not: (a) pregnant, (b) suffering from or diagnosed with hyperthyroidism, (c) suffering from or diagnosed with glaucoma, (d) a nursing mother, or (e) taking monoamine oxidase inhibitors. FIG. 4 shows an example workflow for determining if a patient is an appropriate candidate for the drug and whether additional counseling regarding the potential risks of birth defects is advisable. The patient visits the HCP 401 and a BMI is determined. If the BMI is at least 27 (407), but less than 30, the patient is evaluated to determine if he/she has a weight related co-morbidity 409. If not, the patient is not a good candidate and no prescription is written 411. If yes, or if the patient has a BMI that is at least 30 (403), the patient is evaluated for other contraindications 405. If the patient has other contraindications, the patient is not a good candidate and no prescription is written 427. If the patient has no other contraindications identified in 405, female patients are evaluated for child bearing potential 415, for example, based on age or past surgical procedures. If the patient is a male or there is no child bearing potential, the HCP may determine that it is appropriate to prescribe the drug to the patient 425. If the patient has child bearing potential, the HCP may take additional steps to determine if a prescription might be appropriate. For example, the HCP may determine that the patient is or is not using suitable contraception 413, which may be selected from a list of suitable contraception methods provided by the drug manufacturer in training material provided to the HCP. If the patient is not yet using suitable contraception, the HCP may provide counseling to the patient to initiate suitable contraception 413. If the patient is not using suitable contraception or has not been counseled to initiate suitable contraception, the HCP may determine that the patient should not be prescribed the drug 411. If the patient is using appropriate contraception or has been counseled to initiate suitable contraception, the HCP may counsel the patient on the potential risks for birth defects associated with the drug 423 and then prescribe the drug 425.

In certain embodiments of the invention, the drug containing topiramate may further comprise phentermine. In such embodiments, the drug may be formulated for oral administration as described, for example, in U.S. Pat. No. 7,674,776, the disclosure of which is hereby incorporated herein by reference in its entirety. In accordance with such embodiments, the drug may be formulated as an extended release capsule. In certain embodiments, the drug is formulated for immediate release of phentermine and extended release or controlled release of topiramate. Each dose of drug dispensed to the patient contains from about 20 mg to about 100 mg of topiramate and from about 3 mg to about 15 mg of phentermine. More preferably, topiramate is administered in a dose of from about 23 mg to about 92 mg, with a dose about 46 mg being even more preferred. Dosage forms of the drug can include, but are not limited to, about 3.75 mg phentermine in combination with about 23 mg topiramate, about 7.5 mg phentermine in combination with about 46 mg topiramate, about 11.25 mg phentermine in combination with about 46 mg topiramate, and about 15 mg phentermine in combination with about 92 mg topiramate. For additional information on the use of the combination of topiramate (extended-release) and phentermine for chronic weight management in overweight and obese patients see Allison et al, Obesity; 20(2):330-342 (2011), Gadde et al., Lancet; 377:1341-52 (2011) and Garvey et al., Am J Clin Nutr;95(2):297-308 (2012).

In some embodiments, the methods of the invention comprise contacting prescribers of the drug and providing training to the prescribers. As used herein, the term “prescriber” refers to an individual who is licensed to prescribe drugs, including, but not limited to, nurse practitioners, physician assistants, and medical doctors, such as general practitioners, family practitioners, internists, gynecologists, endocrinologists, and cardiologists and who has written at least one prescription for the drug. Those individuals that are licensed to prescribe the drug, but have not written a prescription for the drug, may be referred to herein as nono-prescribers, potential prescribers, or healthcare providers. The training may employ various educational materials to ensure proper prescribing, dispensing and patient compliance according to the methods described herein, including, for example, a variety of literature and other materials, such as, for example, product information, patient brochures, medication guides, educational brochures, dosing and management checklists, patient counseling checklists, continuing education monographs, videotapes and the like which may describe the risks and benefits associated with taking the particular drug and measures which may be taken to avoid those risks. In some embodiments, the training is provided to prescribers of the drug through a continuously available internet website. In some of such embodiments, the training can be accessed by prescribers of the drug using a unique identifier number, such as a DEA and/or a National Provider Identifier (NPI) number. The prescriber may be registered in a computer readable storage medium, as described in more detail below, following completion of the web-based training and optionally through the prescriber's DEA and/or NPI number.

In certain embodiments, the training comprises providing information regarding the proper use of the drug (e.g., selection of patients, adjustment of dosages and treatment regimens) and/or the risks associated with taking the drug. In some embodiments, the training comprises providing patient brochures and/or medication guides with directions to distribute and review these materials with patients for whom the drug is to be prescribed. In other embodiments, the training comprises directing prescribers to counsel the patient and optionally may include a counseling checklist to assist the prescriber in reviewing all necessary information with the patient. Such counsel may be provided verbally, as well as in written form.

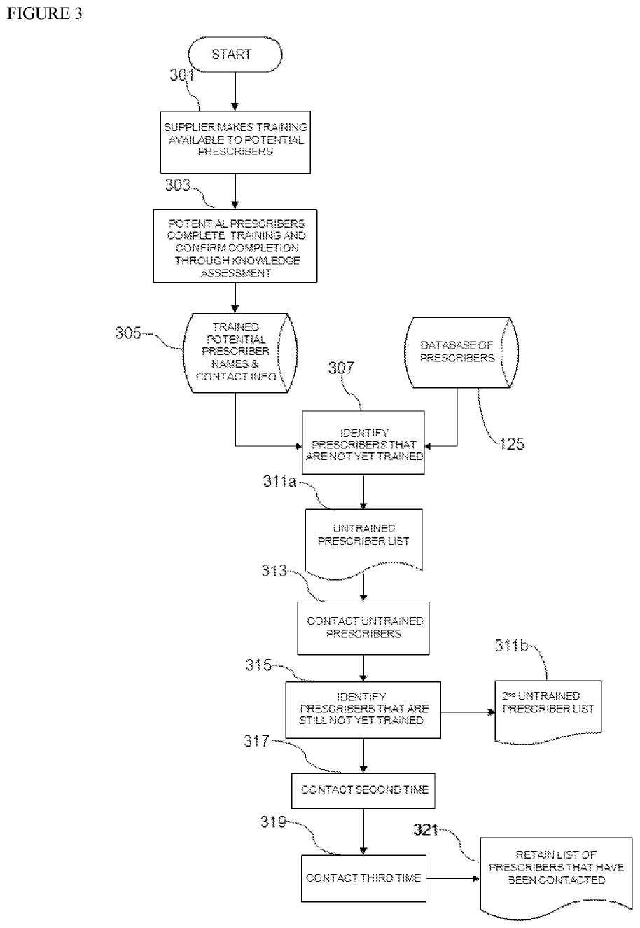

FIG. 3 shows a work flow for a method of providing prescriber training according to one embodiment of the invention. The supplier (e.g. drug manufacturer) makes training available to potential prescribers of the drug (e.g. health care providers) 301. Potential prescribers complete the training and confirm training through a knowledge assessment 303 and provide identifying information such as name, address, contact information (e.g. phone number or email), DEA or NPI number, to the supplier or the supplier's designee to be retained in a database of trained potential prescribers 305. The database of prescribers 125 is compared to the database of trained potential prescribers 305 to identify prescribers that are not yet trained 307. A list of untrained prescribers 311a is produced from the comparison, which is preferably done using a computer. The untrained prescribers on list 311a are contacted by the supplier or supplier's designee 313. The contact includes an offer to provide training and may include, for example, an email with a link to online training or may be in the form of a mailing with training materials included. After a period of time, for example, about 30, 60 or 90 days, the comparison of the trained potential prescriber database 305 is updated to include newly trained individuals and compared a second time (in step 315) to the prescriber database 125. Another list 311b of untrained prescribers is generated and contacted a second time with an opportunity for training 317. Prescribers that are not yet trained are identified and contacted a third time 319 after a determined period of time. Prescribers that have been contacted a third time and remain untrained are retained on a list of untrained prescribers that have been contacted 321. In preferred aspects, steps are taken so that prescribers that are on list 321 are not contacted and offered further training in the future.

In some aspects the training is made available to all potential prescribers regardless of whether they have prescribed the drug to any patients (prescribers and non-prescribers). Training may also be targeted to prescribers that have prescribed the drug to at least one patient. Information about such prescribers sometimes referred to herein as “actual prescribers” may be captured by the certified pharmacies at the time the prescription is submitted to the pharmacy or at the time the prescription is filled. When the training is made available to both prescribers and non-prescribers (e.g. healthcare providers) the trained prescribers (and the trained prescriber database) will include both prescribers and non-prescribers that may be future prescribers, but have not yet written a prescription. A database of trained prescribers may include both prescribers and non-prescribers (healthcare providers). A comparison of a database of prescribers to the trained prescribers will identify prescribers (have written at least one prescription for the drug) that are not yet trained, sometimes referred to as untrained prescribers. In some aspects training is targeted to untrained prescribers.

In some embodiments of the methods of the invention, the training includes a dosing and management checklist to assist the prescriber in, inter alia, identifying proper patients, prescribing an appropriate dosage regimen of the drug, and adjusting dosage during the course of treatment. For instance, in one embodiment, the methods of the invention comprise providing prescribers with a dosing and management checklist that provides instructions for (i) identifying appropriate patients; (ii) prescribing an initial dose of the drug; (iii) counseling the patient on behaviors to be used in combination with the drug; (iv) monitoring the patient during treatment; and (v) evaluating whether a dose escalation is appropriate. In certain embodiments, the initial dose of the drug (e.g. lowest dose) may be prescribed for an initial period, e.g. 14 days. In other embodiments, the dosing and management checklist may provide instructions to the prescriber to prescribe two different doses of the drug: a first initial dose for an initial period and a second dose for a second period (e.g. 30 days). The second dose may be higher than the initial first dose. For example, in certain embodiments, the dosing and management checklist provides instructions for (i) identifying appropriate patients; (ii) prescribing a first 14 day prescription of a first dose of the drug and a second 30 day prescription of a second dose of the drug; (iii) counseling the patient on behaviors to be used in combination with the drug; (iv) monitoring the patient during treatment; (v) determining at 12 weeks if dose escalation is appropriate; and (vi) escalating the patient's dose. In another embodiment, the methods of the invention comprise providing prescribers with a dosing and management checklist that provides instructions for: (i) identifying appropriate patients; (ii) preparing a first prescription for 14 days of the lowest dose of the drug; (iii) preparing a prescription for 30 days of a middle dose of the drug: (iv) instructing the patient to take the drug once daily in the morning with or without food; (v) suggesting follow up communication at between 2 and 8 weeks; (vi) counseling patients to use contraception, cat properly, engage in regular physical activity, not share the drug with any other individual, and report any symptoms of concern to the prescriber; and (vii) monitoring patients for weight and status of comorbidities (e.g., hypertension, type 2 diabetes mellitus, or dyslipidemia). The dosing and management checklist may also include safety information about the drug and instructions to the prescriber for reporting adverse side effects to the drug manufacturer or authorized distributor. Preferably, the dosing and management checklist is in the form of a single document and in some embodiments, does not exceed two pages.

In embodiments in which the drug is a potential teratogenic drug, such as topiramate (Margulis et al., Pharmacoepidemiol Drug Saf (2011) 20 (Suppl 1):S1 1 and Green et al., Headache (2012) 52:1070-1082), the training can include materials which describe the potential risk of birth defects (e.g., orofacial clefts) associated with the drug, the need to counsel female patients about pregnancy prevention, the recommendation for pregnancy testing prior to initiation and periodically during treatment, and the need to discontinue use of the drug if pregnancy occurs, or other such measures directed at minimizing fetal exposure while maintaining access to drug treatment for appropriate patients. In some embodiments, the training comprises directing prescribers to counsel the patient regarding the necessity of pregnancy prevention while taking the drug. The training may also direct prescribers to provide the patient with means of contraception. In certain further embodiments, the training comprises instructing the prescriber to take one or more or all of the following steps with a female patient of reproductive potential, prior to prescribing the drug: (i) notifying the patient that the drug is associated with an increased risk of congenital malformations, such as orofacial clefts, that occur early in pregnancy; (ii) advising the patient that a pregnancy test is recommended before initiating treatment with the drug and periodically (e.g., monthly) during treatment; (iii) advising the patient that if she has a positive pregnancy test she will not be prescribed the drug and if already receiving the drug must stop immediately and report the pregnancy to the prescriber; (iv) provide to the patient a brochure provided by the drug manufacturer disclosing risk of birth defects associated with the drug; and (v) review at least two methods for contraception with the patient.

In particular embodiments, a female patient of reproductive potential is advised to select one method of contraception from a list of highly effective methods of contraception or two methods in combination from a list of methods that are acceptable when used in combination. Highly effective methods of contraception that can be used alone include, but are not limited to, intrauterine device, intrauterine system, progestin implant, tubal sterilization, and male partner's vasectomy. Methods of contraception that are acceptable when used in combination include hormonal contraception and a barrier method. Suitable barrier methods include, but are not limited to, diaphragm with spermicide, cervical cap with spermicide, and a male condom with or without spermicide. Suitable hormonal contraception can be estrogen and progestin combination therapy or progestin monotherapy. In some embodiments, the estrogen and progestin combination therapy is delivered orally (e.g. by a pill), by a transdermal patch, or by a vaginal ring. In other embodiments, the progestin monotherapy is delivered orally (e.g. by a pill) or by injection. Patients are preferably counseled to use contraceptive methods for a period of time prior to and during treatment with the teratogenic drug (e.g., topiramate), as well as for a period of time after treatment with the drug has been terminated. In some embodiments, it may be desirable for the prescriber to personally provide female patients of reproductive potential with one or more contraceptive devices or formulations. In certain embodiments of the invention, the counseling comprises advising the patient to not share the drug with anyone else, and particularly with women of reproductive potential, and that the drug should be kept out of the reach of children.

The training provided to prescribers as described herein can be in electronic or printed form. For instance, in one embodiment, the electronic training comprises (i) individual screens of content that require a user interaction to advance to subsequent screens; and (ii) a plurality of knowledge assessment questions integrated within the training which must be answered correctly by the prescriber prior to completing the training. Electronic training may be available on an internet website, at professional and educational meetings, and through in-person meetings with medical liaisons or representatives of the drug manufacturer. In another embodiment, the printed form of the training comprises a hard copy version of the content provided in the electronic training and a statement of completion containing a plurality of knowledge assessment questions that must be answered correctly by the prescriber in order to complete the training. Printed training is available during prescriber visits, at professional or educational meetings, or upon request from the drug manufacturer via e-mail or mail. Training may be made available using a portable device such as a tablet computer, (for example on an iPad or other similar device) that may be accessed at professional and educational meetings or at in-person meetings between prescribers and representatives of the drug manufacturer. Additional materials that may be made available to the prescribers as part of the training may include a patient brochure describing the risk of birth defects with the drug, prescriber counseling tools for females of reproductive potential and a prescriber dosing and management checklist. These materials may also be made available continuously available to prescribers and healthcare providers on a website dedicated to information about the safe use of the drug. In preferred aspects, a website is provided that is dedicated to the training and educational materials regarding proper use and distribution of the drug as well as information about the drug risks. A call center to provide support to prescribers and patients and a separate call center to support certified pharmacy questions may also be provided.

In a preferred embodiment, prescribers who complete the training, sometimes referred to herein as “trained prescribers” are registered in a computer readable storage medium, such as a database or internet website. In one embodiment, the database of trained prescribers is a secure database being protected by encryption software and/or accessible only by user name and password. The computer readable storage medium in which the trained prescribers are registered may be the same as, or different from the computer readable storage medium in which the certified pharmacies are registered. For prescribers who have been provided with an electronic form of the training, completion of the training occurs when the prescriber views all module training screens and completes correctly a plurality of knowledge assessment questions. For prescribers who have been provided with a printed form of the training, completion of the training occurs when the statement of completion accompanying the training is filled out by the prescriber, including correctly answering the plurality of knowledge assessment questions contained therein, and submitted to the manufacturer or distributor of the drug, or other authorized recipient by, for example, mail or facsimile. In certain preferred embodiments, prescribers who have completed training are registered in the computer readable storage medium using a unique identifier, such as a DEA and/or NPI number. The computer readable storage medium may also contain a registered prescriber's name, contact information, and organization. Other information about the prescriber may also be maintained in the database of trained prescribers.

The present invention also includes methods for identifying prescribers of the drug who have not received training regarding the potential risks associated with the drug. In one embodiment, the method comprises comparing a list of registered prescribers (e.g. trained prescribers) to the list of prescribers maintained by the certified pharmacies to identify unregistered prescribers who have not completed training. Such a comparison between the list of registered prescribers who have received training and the list of prescribers of the drug can be conducted on a periodic basis, such as monthly, quarterly, biannually, and annually. In some embodiments, the unregistered prescribers (i.e. untrained prescribers) are contacted and provided with training or access to training materials. At least 80%, at least 85%, preferably at least 90%, or more preferably at least 95% of the unregistered prescribers (i.e. untrained prescribers) are contacted within thirty days of identification as an unregistered prescriber. Unregistered prescribers may be contacted through electronic communication (e.g. electronic mail), mail, or by phone. In one embodiment, unregistered prescribers are contacted by electronic mail including a link to an electronic form of the training.

Comparisons of the list of registered prescribers (i.e. prescribers who have completed training) to the list of prescribers who have prescribed the drug but who have not completed training (i.e. unregistered prescribers who have not completed training) can be made repeatedly following initial contact with unregistered prescribers in an effort to provide training to all prescribers of the drug. In one embodiment, the methods of the invention comprise preparing a list of the unregistered prescribers who have not completed the training within sixty days of identification as an unregistered prescriber, and sending to each unregistered prescriber on the list a second electronic mail, a printed form of the training, and a letter directing each unregistered prescriber to complete the training. In another embodiment, the methods of the invention comprise preparing a list of the unregistered prescribers who have not completed the training within ninety days of identification as an unregistered prescriber, and sending to each unregistered prescriber on the list a third electronic mail and contacting each unregistered prescriber on the list by phone to provide notification of the availability of training and to encourage the unregistered prescriber to complete the training.

The present invention also includes a method for providing training relating to the safety of a drug to prescribers of the drug. In some embodiments, the drug is a potential teratogenic drug. In one particular embodiment, the drug is topiramate (e.g. extended release form of topiramate). In one embodiment, the method comprises (a) making training available for all prescribers of the drug; (b) maintaining a database of healthcare providers who have completed the training; (c) obtaining a database of prescribers who have prescribed the drug; (d) comparing the database in (b) to the database in (c) to identify prescribers who have prescribed the drug but have not yet completed training thereby identifying untrained prescribers; (e) providing untrained prescribers with a first electronic communication comprising a link to an electronic form of the training; (f) repeating step (d) after a first period of time to identify untrained prescribers that remain untrained; (g) providing untrained prescribers identified in step (f) with a second electronic communication comprising a link to the electronic form of the training and a print form of the training; (h) repeating step (d) after a second period of time to identify untrained prescribers that remain untrained; and (i) providing untrained prescribers identified in step (h) with a third electronic communication comprising a link to the electronic form of the training and contacting the untrained prescriber by phone to inform the prescriber about the availability of the training. The training can include any information as described herein, but preferably includes information relating to adverse side effects and risks associated with the drug. In some embodiments, a record is maintained of each communication to untrained prescribers. A list of prescribers who remain untrained may be maintained. The first period of time can be about 30 days, about 45 days, about 60 days, about 75 days, or about 90 days from the date upon which the untrained prescribers are first identified as untrained prescribers. The second period of time can be about 60 days, about 75 days, about 90 days, about 105 days, or about 120 days from the date upon which the untrained prescribers are first identified as untrained prescribers. In some embodiments, the database of prescribers who have prescribed the drug is established by collecting prescription data from registered pharmacies. Preferably the prescription data comprises the DEA and/or NPI number of the prescriber.

The present invention also encompasses a method for training prescribers of a drug about the potential risks associated with the drug. In some embodiments, the drug is a potential teratogenic drug. In one particular embodiment, the drug is topiramate. The method comprises (a) obtaining a list of potential prescribers of the drug; (b) providing each of the potential prescribers on the list with information related to the risks of the drug; (c) providing training related to the potential risks of the drug to the potential prescribers; (d) monitoring completion of the training to obtain a first database of healthcare providers that have completed the training; (e) obtaining a second database of prescribers who have prescribed the drug; (f) comparing the first database to the second database to identify untrained prescribers; (g) contacting the untrained prescribers to provide training; (h) updating the first database; and (i) repeating steps (e)-(g) at least once. In some embodiments, the risks associated with the drug are risks of birth defects, such as congenital malformations (e.g., orofacial clefts). Preferably, steps (e)-(g) of the method are repeated until at least 80%, at least 85%, at least 90%, at least 95%, or all of the prescribers of the drug have completed the training.

In some embodiments, the present invention provides methods to assess patients' knowledge and understanding of the potential risks of the drug. Periodic surveys may be conducted in a representative sample of patients taking the drug. The patients in the sample are preferably identified as females of reproductive potential and are surveyed for knowledge of the potential risks of the drug for birth defects. The survey may assess patients' understanding of the potential risks of the drug, the extent of counseling regarding pregnancy prevention and contraceptive use that is being provided to females of reproductive potential and the use of contraceptives by females of reproductive potential. A metric may be obtained that represents the percentage of patients who correctly identify the key risk messages. Prescribers and pharmacists may be similarly surveyed to assess knowledge of the potential risks and the effectiveness of training regarding the potential risks.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

TABLE 1
Compliance data requirements to be submitted to supplier
or supplier designee by certified pharmacies weekly

NCPDP#
REMS-specific Patient ID
Prescriber DEA#
Prescriber Last Name
Prescriber First Name
Prescriber Zip Code
Date Rx Received at Certified Pharmacy
New or Refill Script: Yes/No
Fill Date (mmddyy)
Dose/Strength by NDC
Pill Quantity

TABLE 2
Order/inventory data to be gathered by certified
pharmacies at a preferred weekly frequency.

NCPDP#
Purchase/Order Date
Units [bottles] Ordered (by NDC, by date)
Units [bottles] Received (by NDC, by date)
Units [bottles] Dispensed/Shipped to Patients (by NDC)
Inventory On-hand (by NDC, by Lot #)
Returnable Units On Hand (by NDC, by Lot #)
Returns Units Shipped to Supplier (by NDC, by Lot #)

TABLE 3
De-identified longitudinal referral/dispensing data, preferably
provided in weekly data files to supplier or supplier's designee

NCPDP#
Patient ID (to be different from REMS Specific Patient ID, and shall
not be translatable or cross identifiable with REMS specific Patient ID)
Prescriber DEA # Add physician first name and physician last name
Prescriber Last Name
Prescriber First Name
Prescriber Zip
Gender
Age Conversion on day order filled (for Patients aged 89 or younger)
New/Refill Script
Days from Last Fill
Fill Month (mmyy)
Dose/Strength by NDC
Pills Dispensed by NDC
Ship to Zip
Payer Type (Includes Self Pay)
Rx List Price - Self Pay
Commercial Insurance Copay - Out of Pocket ($)
Manufacturer Copay Assistance Applied ($)
Free Goods Script (Y/N)
Medication Possession Ratio (persistency)

1. A method for dispensing a drug containing topiramate to a patient in need of weight loss while minimizing the occurrence of an adverse side effect, the method comprising:
identifying a population of eligible pharmacies that have an established data management system to maintain a list of prescribers of the drug and direct distribution of information related to the risks associated with taking the drug to the patient each time a prescription for the drug is filled;
registering said population of eligible pharmacies in a computer readable storage medium to obtain a record of certified pharmacies;
providing said certified pharmacies with said information related to the potential risks associated with taking the drug; and
authorizing distribution of the drug to said certified pharmacies, wherein said certified pharmacies dispense the drug with said information related to the potential risks associated with taking the drug to said patient pursuant to a prescription.

2. The method of claim 1, wherein said eligible pharmacies agree to train employees on the potential risks associated with taking the drug and the proper measures for dispensing the drug.

3. The method of claim 1, wherein said eligible pharmacies agree to submit to periodic audits of procedures for dispensing the drug.

4. The method of claim 1, wherein said information related to the potential risks associated with taking the drug comprises a medication guide and a patient brochure.

5. The method of claim 4, wherein the medication guide and patient brochure are provided with each prescription and each refill of the drug.

6. The method of claim 4, wherein the certified pharmacies have an automated system that instructs pharmacists and staff dispensing the drug to include the medication guide and patient brochure with each prescription and each refill of the drug.

7. The method of claim 4, wherein the medication guide and patient brochure are also made available on the internet, by calling a toll free number, and by delivery by field-based personnel.

8. The method of claim 4, wherein said patient brochure describes a potential risk of birth defects associated with taking the drug.

9. The method of claim 1, wherein said patient is a female of reproductive potential and said adverse side effect is a birth defect.

10. The method of claim 9, wherein said birth defect is an orofacial cleft.

11. The method of claim 1, wherein said patient has a body mass index of 30 kg/m² or greater.

12. The method of claim 1, wherein said patient has a body mass index of 27 kg/m² or greater.

13. The method of claim 12, wherein said patient has at least one weight-related morbidity factor.

14. The method of claim 13, wherein said at least one weight-related morbidity factor is hypertension, type 2 diabetes mellitus, or dyslipidemia.

15. The method of claim 1, wherein said patient in need of weight loss is not: (a) pregnant, (b) diagnosed with hyperthyroidism, (c) diagnosed with glaucoma, (d) a nursing mother, or (e) taking monoamine oxidase inhibitors currently or within 14 days.

16. The method of claim 1, wherein the drug further comprises phentermine.

17. The method of claim 16, wherein said certified pharmacies dispense the drug in a supply not to exceed thirty days.

18. The method of claim 1, further comprising providing training comprising information relating to potential risks associated with taking the drug to prescribers of the drug through a continuously available internet website.

19. The method of claim 1, further comprising contacting prescribers of the drug; providing training to said prescribers wherein said training comprises information regarding the proper use of the drug and the potential risks associated with taking the drug; and registering in a computer readable storage medium said prescribers who complete the training wherein the prescribers are registered in the computer readable storage medium by a unique identifier.

20. The method of claim 19, wherein said training comprises directing prescribers to counsel the patient regarding the necessity of pregnancy prevention while taking the drug and directing prescribers to provide the patient with means of contraception if applicable.

21. The method of claim 20, wherein said training comprises instructing the prescriber to take the following steps with a female patient of reproductive potential, prior to prescribing the drug:
(i) notifying the patient that the drug is associated with an increased risk of congenital malformations including orofacial clefts that may occur early in pregnancy;
(ii) advising the patient that a pregnancy test is recommended before initiating treatment with the drug and monthly during treatment;
(iii) advising the patient that if she has a positive pregnancy test she will not be prescribed the drug and if already receiving the drug must stop immediately and report the pregnancy to the prescriber;
(iv) providing to the patient a brochure provided by the drug manufacturer disclosing potential risk of birth defects associated with the drug; and
(v) reviewing effective methods for contraception with the patient.

22. The method of claim 21, wherein the prescriber counsels the patient in the selection of at least one method of contraception from a list of highly effective single methods of contraception or two methods in combination from a list of methods that are acceptable when used in combination, wherein the highly effective single methods of contraception are selected from the list consisting of intrauterine device, intrauterine system, progestin implant, tubal sterilization and male partner's vasectomy.

23. The method of claim 22, wherein the methods that are acceptable when used in combination are selected from the list consisting of hormonal contraception and a barrier method selected from diaphragm with spermicide, cervical cap with spermicide, and a male condom with or without spermicide.

24. The method of claim 23, wherein the hormonal contraception method is estrogen and progestin combination therapy or progestin monotherapy.

25. The method of claim 19, wherein said training is electronic training or printed training accompanied by a statement of completion wherein the electronic training comprises (i) individual screens of content that require a user interaction to advance to subsequent screens; and
(ii) a plurality of knowledge assessment questions integrated within the training which must be answered correctly by the prescriber prior to completing the training.

26. The method of claim 19, further comprising comparing a list of registered prescribers to the list of prescribers maintained by the certified pharmacies to identify unregistered prescribers who have not completed training.

27. The method of claim 26, further comprising contacting said unregistered prescribers and offering training to said unregistered prescribers.

28. The method of claim 27, wherein at least 95% of the unregistered prescribers are contacted within thirty days of identification as an unregistered prescriber.

29. The method of claim 27, wherein the unregistered prescribers are contacted by a first electronic mail including a link to an electronic form of the training and further comprising preparing a list of the unregistered prescribers who have not completed the training within sixty days of identification as an unregistered prescriber, and sending to each unregistered prescriber on the list a second electronic mail, a printed form of the training, and a letter directing each unregistered prescriber to complete the training.

30. The method of claim 29, further comprising preparing a list of the unregistered prescribers who have not completed the training within ninety days of identification as an unregistered prescriber, and sending to each unregistered prescriber on the list a third electronic mail and contacting each unregistered prescriber on the list by phone to provide notification of the availability of training and to encourage the unregistered prescriber to complete the training.

31. The method of claim 19, wherein the training is provided on an internet website, at meetings, or by medical liaisons and wherein completion of the program is recorded on a statement of completion that is submitted by the prescriber to confirm training.

32. The method of claim 19, wherein the prescriber completes the training electronically by viewing all module training screens and completing a plurality of knowledge assessment questions.

33. The method of claim 19, wherein the unique identifier is a Drug Enforcement Administration (DEA) or National Provider Identifier (NPI) number.

34. The method of claim 19, further comprising providing prescribers with a dosing and management checklist, in the form of a single document, that provides instructions for (i) identifying appropriate patients; (ii) prescribing one or more doses of the drug; (iii) counseling the patient on behaviors to be used in combination with the drug; (iv) monitoring the patient during treatment; (v) determining at 12 weeks if dose escalation is appropriate; and (vi) escalating the patient's dose.

35. The method of claim 34, wherein the dosing and management checklist provides instructions for prescribing a first prescription of a first dose of the drug and a second prescription of a second dose of the drug.

36. The method of claim 19, further comprising providing prescribers with a dosing and management checklist, in the form of a single document, that provides instructions for: (i) identifying appropriate patients; (ii) preparing a first prescription for the lowest dose of the drug; (iii) preparing a second prescription for a middle dose of the drug: (iv) instructing the patient to take the drug once daily in the morning with or without food; (v) suggesting follow up communication at between 2 and 8 weeks; (vi) counseling patients to use contraception, eat properly, engage in regular physical activity, not share the drug with any other individual, and report any symptoms of concern to the prescriber; and (vii) monitoring patients for weight and status of comorbidities.

37. The method of claim 34, wherein the dosing and management checklist further comprises safety information about the drug and instructions to the prescriber for reporting adverse side effects to the drug manufacturer.

38. The method of claim 1, wherein said certified pharmacies do not resell or transfer the drug to non-certified pharmacies.

39. The method of claim 1, wherein the certified pharmacies dispense the drug to the patient through certified retail dispensing locations or by mail order.

40. The method of claim 1, wherein the drug is formulated for oral administration as an extended release capsule and wherein each dose of the drug contains about 20 mg to about 100 mg of topiramate.

41. A method for providing training relating to the safety of a drug to prescribers of the drug comprising:
(a) making training available for all prescribers of the drug;
(b) maintaining a database of healthcare providers who have completed the training;
(c) obtaining a database of prescribers who have prescribed the drug;
(d) comparing the database in (b) to the database in (c) to identify prescribers who have prescribed the drug but have not completed training thereby identifying untrained prescribers;
(e) providing untrained prescribers with a first electronic communication comprising a link to an electronic form of the training;
(f) repeating step (d) after a first period of time to identify untrained prescribers that remain untrained;
(g) providing untrained prescribers identified in step (f) with a second electronic communication comprising a link to the electronic form of the training and a print form of the training;
(h) repeating step (d) after a second period of time to identify untrained prescribers that remain untrained; and
(i) providing untrained prescribers identified in step (h) with a third electronic communication comprising a link to the electronic form of the training and contacting the untrained prescriber by phone to inform the prescriber about the availability of the training.

42. The method of claim 41, wherein a record is maintained of each communication to untrained prescribers.

43. The method of claim 41, wherein the first period of time is about 30 days from the date upon which the untrained prescribers are first identified as untrained prescribers and the second period of time is about 60 days from the date upon which the untrained prescribers are first identified as untrained prescribers.

44. The method of claim 41, wherein the first period of time is about 60 days from the date upon which the untrained prescribers are first identified as untrained prescribers and the second period of time is about 90 days from the date upon which the untrained prescribers are first identified as untrained prescribers.

45. A method for training prescribers of a drug about the potential risks associated with the drug comprising:
(a) obtaining a list of potential prescribers of the drug;
(b) providing each of the potential prescribers on the list with information related to the potential risks of the drug;
(c) providing training related to the potential risks of the drug to the potential prescribers;
(d) monitoring completion of the training to obtain a first database of healthcare providers that have completed the training;
(e) obtaining a second database of prescribers who have prescribed the drug;
(f) comparing the first database to the second database to identify untrained prescribers;
(g) contacting the untrained prescribers to provide training;
(h) updating the first database; and
(i) repeating steps (e)-(g) at least once.
